(19) United States
(12) Patent Application Publication
(10) Pub. No.: US 2022/0143153 A1
(43) Pub. Date: May 12, 2022

(54) COMPOSITIONS AND METHODS FOR TREATING ACNE

(71) Applicant: The Regents of the University of California, Oakland, CA (US)

(72) Inventors: Tran DO, Los Angeles, CA (US); Robert L. MODLIN, Sherman Oaks, CA (US); Peter C. DEDON, Boston, MA (US)

(73) Assignee: The Regents of the University of California, Oakland, CA (US)

(21) Appl. No.: 17/435,541

(22) PCT Filed: Mar. 9, 2020
(86) PCT No.: PCT/US2020/021721

§ 371 Date: Sep. 1, 2021

Related U.S. Application Data
(60) Provisional application No. 62/815,542, filed on Mar. 8, 2019.

(51) Int. Cl. A61K 38/46; A61K 8/66; A61K 9/00; A61P 17/10; A61Q 19/00

(52) U.S. Cl. CPC A61K 38/465; A61K 8/66; C12Y 301/27009; A61P 17/10; A61Q 19/00; A61K 9/0014

(57) ABSTRACT

The invention relates to compositions and methods for treating an acne. In one embodiment, the invention relates to the use of an RNA inhibitor to treat an acne.

### CROSS-REFERENCE TO RELATED APPLICATIONS

This application claims the benefit of U.S. Provisional Application U.S. Ser. No. 62/815,542, filed Mar. 8, 2019, which is incorporated in its entirety herein by reference.

### GOVERNMENT SUPPORT STATEMENT

This invention was made with government support under Grant Number AR074302, awarded by the National Institutes of Health. The government has certain rights in the invention.

### FIELD OF THE INVENTION

The invention relates to compositions and methods for treating an acne. In one embodiment, the invention relates to the use of an RNA inhibitor to treat an acne.

### BACKGROUND OF THE INVENTION

Microbes play a large part in the pathogenesis of disease. On the skin surface, the interplay within the microbial community is essential for the maintenance of healthy skin. Cutibacterium acnes (formerly known as Propionibacterium acnes) is a Gram-positive, microaerophilic bacillus that is considered to be one of the factors driving inflammation in acne. However, the direct cause-and-effect relationship between the bacteria has been difficult to establish given that C. acnes is a ubiquitous bacterium and that there was no quantitative difference in the number of bacteria between subjects with and without acne.

Recent genomic and phenotypic analyses provide new insights on the cellular physiology underlying the heterogeneity of the bacteria. Certain C. acnes strains, designated type IA₁ or IC, identified by multi-locus sequence typing (MLST) were found to be strongly associated with acne (C_{A}), while phylotype II strains were preferentially present on the skin of subjects with healthy or clear skin (C_{H}). A more comprehensive metagenomic analysis using ribotyping found that acne-associated types were present in significant quantities in approximately 30-40% of patients with acne but rarely in individuals with healthy skin. Conversely, the phylotype II, RT 6 subgroup was found to be 99% associated with healthy skin. The two divergent phylotypes also exhibit differences in inflammatory potential with C_{A} inducing higher inflammatory cytokine secretion, such as IFN-γ and IL-17, from human peripheral blood mononuclear cells (PBMCs) while C_{H} induces higher anti-inflammatory IL-10.

Given the increasing recognition that commensal and mutualistic microorganisms are necessary for the maintenance of a healthy human physiology, it is essential to understand how the different phylotypes in C. acnes modulate the inflammatory microenvironment.

Accordingly, there exists a need for improved compositions and methods for treating an acne by modulating its host-microbiome interactions.

### SUMMARY OF THE INVENTION

In one aspect, the invention provides a method for treating an acne in a subject in need thereof, the method comprising: administering to said subject a therapeutically effective amount of an RNA inhibitor, thereby treating said acne in said subject. In an exemplary embodiment, the RNA inhibitor is an RNAse (e.g., t-RNAse).

In another aspect, the invention provides a method for inhibiting or inactivating a virulent strain of acne bacterium in a subject in need thereof, the method comprising: administering to said subject a therapeutically effective amount of an RNA inhibitor, thereby inhibiting or inactivating said virulent strain of acne bacterium in said subject.

In yet another aspect, the invention provides a method for eliciting an anti-inflammatory response in a subject in need thereof, the method comprising: administering to said subject a therapeutically effective amount of an RNA inhibitor, thereby eliciting said anti-inflammatory response in said subject. In one embodiment, the anti-inflammatory response is elicited by downregulating the expression of IFN-γ, TNF-α, IL-1β, IL-6, IL-8, IL-17, IL-18, IL-18R, TLR-8 or any combination thereof. In another embodiment, the anti-inflammatory response is elicited by upregulating the expression of IL-10.

In yet another aspect, the invention provides a method for treating a skin inflammation induced by Cutibacterium acnes in a subject in need thereof, the method comprising: administering to said subject a therapeutically effective amount of an RNA inhibitor, thereby treating said skin inflammation in said subject.

In another embodiment, the present disclosure provides use of a composition for the preparation of medicament to treat acne in a subject in need thereof, the composition comprises a therapeutically effective amount of an RNA inhibitor.

In another embodiment, the present disclosure provides use of a composition for the preparation of medicament to inhibit or inactivate a virulent strain of acne bacterium in a subject in need thereof, the composition comprises a therapeutically effective amount of an RNA inhibitor.

In another embodiment, the present disclosure provides use of a composition for the preparation of medicament to elicit an anti-inflammatory response in a subject in need thereof, the composition comprises a therapeutically effective amount of an RNA inhibitor.

In another embodiment, the present disclosure provides use of a composition for the preparation of medicament to treat a skin inflammation induced by Cutibacterium acnes in a subject in need thereof, the composition comprises a therapeutically effective amount of an RNA inhibitor.

In one embodiment, the RNA inhibitor in the methods or uses mentioned above is co-administered to a subject with an agent that downregulates the expression and/or function of one or more of IFN-γ, TNF-α, IL-1β, IL-6, IL-8, IL-17, IL-18, IL-18R, and TLR-8.

In a further aspect, the invention provides a pharmaceutical composition comprising a therapeutically effective amount of an RNA inhibitor, wherein said RNA inhibitor is present in an amount effective to treat an acne in a subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIGS. 1A-1D: PBMCs were stimulated with C. acnes C_{A} or C_{H} at MOI 0.5. Various cytokines were measured at 24 h via ELISA.

FIG. 2: PBMCs were stimulated with C. acnes or tetanus toxoid, IFN-γ and IL-10 were measured at various time points via ELISA.

FIGS. 3A-3D: PBMCs were stimulated with untreated, heat-killed (HK), Rnase I, or Dnase I treated live C_{A}(HL5PA1), C_{H} (HL110PA4), LPS, or 19 kD (TLR1/2L) for 24 h. Cytokine secretions were measured by ELISA.

FIGS. 4A-4B: PBMCs were stimulated with increasing concentrations of C_{A} (HL5PA1) total RNA. Cytokines were measured at 24 h via ELISA.

FIGS. 5A-5B: PBMCs stimulated with live, total RNA C_{A} vs. C_{H}, and various TLR ligands. Cytokine were measured at 24 h via ELISA.

FIG. 6: MDMs were stimulated with either live C. acnes or C. acnes total RNA (5 μg/mL) for 24 h. Cytokines were measured by ELISA.

FIGS. 7A-7C: Bioanalyzer analysis of total RNA using the RNA 6000.

FIG. 8A shows UMAP visualization of cell types detected. FIG. 8B shows UMAp visualization of lesional and nonlesional cells distribution with red as lesional cells and turquoise as nonlesional cells.

FIGS. 9A-9F: UMAP visualization of IL1B, IL6, CXCL8, IL18, TNF, and IFNG cytokine gene expression with red scale increases with expression level.

FIG. 10A shows UMAP visualization of myeloid subcluster and the myeloid cell types detected. FIG. 10B shows UMAp visualization of lesional and nonlesional cells distribution in myeloid cells with red as lesional cells and turquoise as nonlesional cells.

FIG. 11A shows violin plot of TLR8 expression in various cell types. FIG. 11B shows UMAP visualization of the TLR8 expression. FIG. 11C shows violin plot of TLR8 expression in myeloid cell subsets. FIG. 11D shows UMAP visualization of the TLR8 expression. FIG. 11E shows violin plot of IL18 expression in myeloid cell subsets. FIG. 11F shows UMAP visualization of IL18 expression. Red scale increases with expression level.

FIG. 12 shows UMAP visualization of IFNG gene expression in lymphoid subcluster (left panel), and UMAP visualization of lymphoid subcluster and the lymphoid cell types detected (right panel).

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament” are used interchangeably herein, as context dictates, to refer to a compound or compounds or composition of matter which, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment with a pharmaceutical composition in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian. In another embodiment, the subject is any organism susceptible to an acne or a skin inflammation.

Conditions and disorders in a subject for which a particular drug or compound or composition (or combination thereof) is said herein to be “indicated” are not restricted to conditions and disorders for which that drug or compound or composition has been expressly approved by a regulatory authority, but also include other conditions and disorders known or reasonably believed by a physician to be amenable to treatment with that drug or compound or composition or combination thereof.

Surprisingly and unexpectedly, the inventors of the instant application found that C. acnes activates the innate immune system through RNA species that are usually reserved for viral detection. The inventors found that RNA species from C_{A} and C_{H} have different bioanalyzer profiles and can trigger distinct immune response as seen with live bacteria. In addition, the inventors found that that RNA from C. acnes can act as the microbial virulence factor activating a robust immune response. As a result, the inventors found that that the RNA from C. acnes can be inhibited by the use of an RNA inhibitor in order to treat acne.

In one embodiment, provided herein is a method for treating an acne in a subject in need thereof, the method comprising: administering to said subject a therapeutically effective amount of an RNA inhibitor, thereby treating said acne in said subject. In an exemplary embodiment, the RNA inhibitor is a ribonuclease (RNase).

In another embodiment, provided herein is a method for inhibiting or inactivating a virulent strain of acne bacterium in a subject in need thereof, the method comprising: administering to said subject a therapeutically effective amount of an RNA inhibitor, thereby inhibiting or inactivating said virulent strain of acne bacterium in said subject.

In yet embodiment, provided herein is a method for eliciting an anti-inflammatory response, in a subject in need thereof, the method comprising: administering to said subject a therapeutically effective amount of an RNA inhibitor, thereby eliciting said anti-inflammatory response in said subject. In some embodiments, the anti-inflammatory response is elicited by downregulating the expression of IFN-γ, TNF-α, IL-1β, IL-6, IL-8, IL-17, IL-18, IL-18R, TLR-8, or any combination thereof. In other embodiments, the anti-inflammatory response is elicited by upregulating the expression of IL-10.

In yet another embodiment, provided herein is a method for treating a skin inflammation, induced by Cutibacterium acnes, in a subject in need thereof, the method comprising: administering to said subject a therapeutically effective amount of an RNA inhibitor, thereby treating said skin inflammation in said subject.

### RNA Inhibitor

In some embodiments, the compositions or formulations described herein comprise an RNA inhibitor. In one embodiment, the RNA inhibitor is RNase (e.g., RNase I). RNase is well known in the art and fully described in, for example, U.S. Pat. Nos. 8,748,572; 6,936,432; 6,855,530; 6,737,572; and 6,214,805, which are incorporated by reference herein in their entirety.

RNase (also referred to herein as ribonuclease, RNases or RNase compound) refers to an enzyme that catalyzes the hydrolysis of ribonucleic acid (RNA), and mediates degradation of RNA.

In one embodiment, RNase is an endoribonuclease. In another embodiment, RNase is an exoribonuclease. Examples of endoribonucleases include, for example, but not limited to, RNase A, RNase H, RNase III, RNase L, RNase P, RNase PhyM, RNase T1, RNase T2, RNase U2, RNase V, nuclease P1 and micrococcal nuclease. Examples of exoribonuclease include, for example, but not limited to PNPase, RNase PH, RNase R, RNase D, RNase T, oligoribonuclease, exoribonuclease I, and exoribonuclease II.

RNases, including endoribonucleases and exoribonucleases, fall into multiple subclasses of the enzyme class EC 3.1 (Ramos-Nino, Drugs of the Future 2007, 32:517-526). Both endogenous and exogenous RNases can be used to mediate cellular toxicity. The use of RNases in therapeutics is fully described in U.S. Patent Application Publications 2005/0261232; 2017/0296647; 2016/0361392; 2016/0045574; 2016/0045431; and 2013/0209443.

One of the earliest RNases for which cytotoxicity was investigated was bovine pancreatic RNase A (Ledoux, et al., Experientia 1954 10(12):500-1, Ledoux, Nature 1955, 175(4449):258-9; Ledoux, Nature 1955, 176(4470):36-7). Subsequently, a higher level of cytotoxicity than that exhibited by RNase A, was shown with two other classes of RNases: BS-RNases, isolated from bovine seminal vesicles (Matousek, Comp Biochem Physiol C Toxicol Pharmacol 2001, 129(3):175-91, Hosokawa et al., J Biochem (Tokyo) 1971, 69(4):683-97, Dostal et al., J Reprod Fertil 1973, 33(2):263-74, Matousek et al., Comp Biochem Physiol A 1973, 46(2):241-8, D'Alessio et al., FEBS Lett 1972, 27(2):285-8, Matousek, Experientia 1973, 29(7):858-9), and RNases derived from the eggs and embryos of frogs (Sakakibara et al., Biochim Biophys Acta 1976, 444(2):386-95, Nitta et al., Cancer Res 1994, 54(4):928-34, Ardelt et al., J Biol Chem 1991, 266(1):245-51, Darzynkiewicz et al., Cell Tissue Kinet 1988, 21:169-82). Ranpirnase refers to an RNase extracted from Rana pipiens, the Northern leopard frog, and has the registered trademark name ONCONASE (U.S. Pat. No. 5,559,212).

In a particular embodiment, the RNA inhibitor is an endonuclease capable of cleaving transfer RNA (tRNA or t-RNA) in a virulent strain of acne bacterium. In one embodiment, RNA inhibitor is tRNase, for example, tRNase Z. In one embodiment, RNA inhibitor is a VapC toxin, which is well known in the art and fully described in, for example, Walling et al., 2018, Journal of Bacteriology, vol. 200 (3), pages e00582-17 and U.S. Patent Application Publication 20150023983.

In some embodiments, the RNA inhibitor is an endonuclease capable of cleaving a small RNA, 16S ribosomal RNA, 23S ribosomal RNA, long noncoding RNA, snoRNA, or any other form of RNA in a virulent strain of acne bacterium.

It should be appreciated that RNases from multiple sources are compatible with methods of the claimed invention. In some embodiments, the RNase is derived from frogs, such as the genus Rana, including Rana pipiens. In certain embodiments, the RNase is ranpirnase. In other embodiments, the RNase is derived from fungi, such as RNase T1 from Aspergillus oryzae and nuclease P1 from Penicillium citrinum. In other embodiments, the RNase is derived from bacteria, such as micrococcal nuclease from Staphylococcus aureus. In other embodiments, the RNase is a mammalian RNase such as a bovine RNase. In some embodiments, the RNase is a human RNase. Human RNases can be modified such that their activities will not be inhibited in human cells, an approach that is discussed further in U.S. Pat. Nos. 5,389,537, 6,280,991, 5,840,296, and U.S. Patent Application Publication 20070003537. In some embodiments of the invention, the RNase is purified from an animal or human tissue, while in other embodiments the RNase is expressed and purified as a recombinant protein in bacteria, discussed further in U.S. Patent Application Publications 20030027311 and 20050014161. RNases consistent with the invention include variants, such as RNases in which the sequence has been modified from its naturally occurring sequence. In some embodiments, the sequence of the RNase is modified to target the RNase to a cancer cell. Targeting of RNases is discussed further in U.S. Pat. No. 6,175,003.

The invention also encompasses functional fragments and variants RNases described herein.

In one aspect, the invention relates to the use of an RNA inhibitor to inhibit or inactivate RNA in a virulent strain of acne bacterium in order to treat acne. In some embodiments, the invention relates to the use of antagonists of IFN-γ, TNF-α, IL-1β, IL-6, IL-8, IL-17, IL-18, IL-18R, TLR-8, or any combination thereof in order to treat acne. In other embodiments, the invention relates to the use of agonists of IL-10 in order to treat acne.

### Pharmaceutical Compositions

In another embodiment, provided herein is a pharmaceutical composition to treat an acne in a subject, comprising: a therapeutically effective amount of a molecule of the invention, wherein said molecule is present in an amount effective to treat said acne.

The invention also provides a pharmaceutical composition comprising an RNA inhibitor and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Moreover, “Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

In an embodiment, pharmaceutical compositions containing the therapeutic agent or agents described herein, can be, in one embodiment, administered to a subject by any method known to a person skilled in the art, such as, without limitation, topically, transdermally, injectably, orally, parenterally, transmucosally, subcutaneously, intramuscularly, intravenously, intraarterially, intra-peritonealy, intra-cranially, or intra-vaginally. In one embodiment, the therapeutic agent or combination of therapeutic agents is administered intra-tumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Saline solutions and aqueous dextrose and glycerol solutions can also be employed as liquid carriers, particularly for injectable solutions.

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Within the present invention, in one embodiment, the molecules of the invention may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine Common salt-forming cations include, without limitation, ammonium, calcium, iron, magnesium, potassium, pyridinium, quaternary ammonium, sodium, and copper. Common salt-forming anions include, without limitation, acetate, carbonate, chloride, citrate, cyanide, fluoride, nitrate, nitrite, oxide, phosphate, and sulfate.

Molecules of the invention can also be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic molecules of the present invention may exist as zwitterions. All forms of the compounds, including free acid, free base and zwitterions, are contemplated to be within the scope of the present invention. It is well known in the art that molecules containing both amino and carboxy groups often exist in equilibrium with their zwitterionic forms. Thus, any of the molecules described herein that contain, for example, both amino and carboxy groups, also include reference to their corresponding zwitterions.

In one embodiment, the compositions are formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

Administration can be systemic or local. It may be desirable to administer a pharmaceutical composition of the invention locally to the area in need of treatment; this may be achieved by, for example, and not by way of limitation, local infusion, by injection, by means of a catheter, by means of a suppository, or by means of an implant, said implant being of a porous, non-porous, or gelatinous material. According to some embodiments, administration can be by direct injection e.g., via a syringe, at the site of an acne.

A molecule of the present invention can be delivered in an immediate release or in a controlled release system. In one embodiment, an infusion pump may be used to administer a molecule of the invention. In another embodiment, a molecule of the invention is administered in combination with a biodegradable, biocompatible polymeric implant, which releases the compound over a controlled period of time at a selected site. Examples of polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

The pharmaceutical compositions of the invention may be formulated in a variety of ways, including for example, solid, semi-solid and liquid dosage forms, such as tablets, pills, powders, capsules, gels, liquid solutions (e.g., injectable and infusible solutions), dispersions or suspensions, liposomes and suppositories. In a particular embodiment, the composition is in the form of a topical gel or a cream. The composition can also be in a form suitable for oral, intravenous, intraarterial, intramuscular, subcutaneous, parenteral, transmucosal, transdermal, or topical administration.

### Effective Doses

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including cancer, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test bioassays or systems.

Moreover, suitable doses may also be influenced by permissible daily exposure limits of any compound included in a formulation or method as described herein. Such limits are readily available, including, for example, from industry guidance recommendations provided periodically from the U.S. Food and Drug Administration, and the evaluation of these limits are within the knowledge and understanding of one of ordinary skill in the art.

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

As used herein, the term “administering” refers to bringing in contact with a compound of the present invention. Administration can be accomplished to cells or tissue cultures, or to living organisms, for example humans. In one embodiment, the present invention encompasses administering the compositions of the present invention to a human subject.

In one embodiment, methods of the present invention comprise the step of contacting one or more cells of said subject with a composition as described herein. In one embodiment, contacting one or more cells of a subject with a composition described herein.

In an embodiment, any of the therapeutic or prophylactic drugs or compositions described herein may be administered simultaneously. In another embodiment, they may be administered at different timepoint than one another. In one embodiment, they may be administered within a few minutes of one another. In another embodiment, they may be administered within a few hours of one another. In another embodiment, they may be administered within 1 hour of one another. In another embodiment, they may be administered within 2 hours of one another. In another embodiment, they may be administered within 5 hours of one another. In another embodiment, they may be administered within 12 of one another. In another embodiment, they may be administered within 24 hours of one another.

In one embodiment, any of the therapeutic or prophylactic drugs or compositions described herein may be administered at the same site of administration. In another embodiment, they may be administered at different sites of administration.

It is to be noted that dosage values and amounts and ratios of individual components of the compositions described herein also may vary with the type and severity of the condition to be alleviated and other factors. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions described and contemplated herein can be included in a container, pack, or dispenser together with instructions for administration.

### Acne, and Other Disorders

The disease or disorder treated by the invention includes, for example, acne or other skin inflammation disorders.

In one embodiment, the acne is Acne vulgaris. In another embodiment, the acne is Acne inversa. In yet another embodiment, the acne is Acne rosacea.

Acne or its inflammation can be induced by Cutibacterium acnes, Staphylococcus epidermidis, Cutibacterium granulosum, Cutibacterium humerusii, or a combination thereof.

Examples of a skin inflammation disorder includes, for example, but not limited to, dermatitis, eczema, and psoriasis.

### Combined Treatments

In another aspect, the invention relates a combination therapy for treating an acne or a skin inflammation. In some embodiments, any of the methods of the invention may comprise administering an RNA inhibitor in combination with one or more therapeutically effective agents or treatments.

Other therapeutically effective agents/treatments include benzoyl peroxide, a retinoid, an antibiotic, a hormonal agent, azelaic acid, salicylic acid, comedo extraction, light therapy, dermabrasion, microneedling. chemical peel, or a combination thereof.

Examples of a retinoid include, for example, but not limited to, include adapalene, isotretinoin, retinol, tazarotene, and tretinoin. Examples of an antibiotic include, for example, but not limited to, include clindamycin, erythromycin, metronidazole, sulfacetamide, and tetracyclines such as doxycycline and minocycline. Examples of a hormonal agent include, for example, but not limited to, estrogen, progestins (e.g., desogestrel, dienogest, drospirenone, or norgestimate), and anti-androgens (cyproterone acetate and spironolactone, flutamide, or clascoterone).

The administration of the RNA inhibitor with other agents and/or treatments may occur simultaneously, or separately, via the same or different route, at the same or different times. Dosage regimens may be adjusted to provide the optimum desired response (e.g., a therapeutic or prophylactic response).

Each of the above additives, excipients, formulations and methods of administration represents a separate embodiment of the present invention.

In another aspect, the invention relates to a cosmetic composition to treat an acne or a skin inflammation in a subject, comprising: a therapeutically effective amount of an RNA inhibitor, wherein said RNA inhibitor is present in an amount effective to treat an acne or a skin inflammation. In some embodiments, the cosmetic composition may include carriers or other ingredients described herein.

In another aspect, the invention provides a kit. A kit is typically packaged individually in a container. A kit may include each of the inventive therapy components described herein premeasured and/or mixed together in a fashion convenient for administration, e.g., formulated into one or more gels, creams, capsules, tablets, syrup, transdermal patches, etc. The kit typically includes instructions for use, which may be on a separate piece of medium (e.g., on a sheet of paper), or printed upon a container itself, or on the surface of a package. Alternatively, or in addition, the instructions may be made available separately via, for example, online sources. The kit comprises at least one unit dosage form of the pharmaceutical composition. Typically, however, the kit contains a supply of the inventive therapy to be taken for a predetermined duration of time, e.g., a 7-day supply, 14-day supply, 30-day supply, 60-day supply, or 90-day supply of the inventive therapy.

In some embodiments, the kit of the invention also includes prescribing information.

The methods of treatment described herein can be used to treat any suitable mammal, including primates, such as monkeys and humans, horses, cows, cats, dogs, rabbits, and rodents such as rats and mice. In one embodiment, the mammal to be treated is human.

In one embodiment, the present disclosure provides a method for treating acne in a subject in need thereof, the method comprises administering to the subject a therapeutically effective amount of an RNA inhibitor, thereby treating the acne in the subject. In one embodiment, the RNA inhibitor inhibits or degrades RNA in a virulent strain of acne bacterium.

In one embodiment, the RNA inhibitor elicits an anti-inflammatory response. In one embodiment, the anti-inflammatory response is elicited by downregulating the expression of IFN-γ, TNF-α, IL-1β, IL-6, IL-8, IL-17, IL-18, IL-18R, TLR-8, or any combination thereof. In another embodiment, the anti-inflammatory response is elicited by upregulating the expression of IL-10.

In one embodiment, the RNA targeted in the above method can be a small RNA, a messenger RNA (mRNA), a ribosomal RNA (rRNA), a transfer RNA (tRNA), a small nuclear RNA (snRNA), a regulatory RNA, a transfer-messenger RNA (tmRNA), a double-stranded RNA (dsRNA), or a combination thereof. In one embodiment, the RNA is 16S ribosomal RNA or 23S ribosomal RNA.

In one embodiment, the RNA inhibitor used in the above method is RNase. In one embodiment, the RNA inhibitor can be RNase I, RNase A, RNase H, RNase III, RNase L, RNase P, RNase PhyM, RNase T1, RNase T2, RNase U2, RNase V, nuclease P1, micrococcal nuclease, PNPase, RNase PH, RNase R, RNase D, RNase T, oligoribonuclease, exoribonuclease I, or exoribonuclease II. In another embodiment, the RNA inhibitor is an endonuclease capable of cleaving tRNA in a virulent strain of acne bacterium. In another embodiment, the RNA inhibitor is tRNase or tRNA cleaving RNAse. In yet another embodiment, the RNA inhibitor is VapC toxin.

In one embodiment, the RNA inhibitor is administered by topical administration, trans-dermal administration, or subcutaneous administration. In another embodiment, the RNA inhibitor is co-administered with another agent. In one embodiment, this another agent can be an acne treating agent. Examples of an acne treating agent include, but are not limited to, benzoyl peroxide, a retinoid, an antibiotic, a hormonal agent, azelaic acid, salicylic acid, or a combination thereof. In one embodiment, the RNA inhibitor is administered independently from this another agent. In another embodiment, the RNA inhibitor is administered in combination with an acne treatment procedure. The acne treatment procedure can be comedo extraction, light therapy, dermabrasion, microneedling. chemical peel, or a combination thereof.

In one embodiment, the acne treated in the above method can be Acne vulgaris, Acne inversa, or Acne rosacea. In another embodiment, the acne is associated with Cutibacterium acnes, Staphylococcus epidermidis, Cutibacterium granulosum, or Cutibacterium humerusii. In one embodiment, the subject treated in the above method is a mammal. In another embodiment, the subject is a human.

In another embodiment, the present disclosure also provides a pharmaceutical composition comprising a therapeutically effective amount of an RNA inhibitor, wherein the RNA inhibitor is present in an amount effective to treating an acne in a subject.

In another embodiment, the present disclosure also provides a method for treating a skin inflammation induced by Cutibacterium acnes in a subject in need thereof, the method comprises administering to the subject a therapeutically effective amount of an RNA inhibitor, thereby treating the skin inflammation in the subject.

In another embodiment, the present disclosure provides use of a composition for the preparation of medicament to treat acne in a subject in need thereof, the composition comprises a therapeutically effective amount of an RNA inhibitor. In one embodiment, the RNA inhibitor inhibits or degrades RNA in a virulent strain of acne bacterium. In one embodiment, the RNA inhibitor elicits an anti-inflammatory response. In one embodiment, the anti-inflammatory response is elicited by downregulating the expression of IFN-γ, TNF-α, IL-1β, IL-6, IL-8, IL-17, IL-18, IL-18R, TLR-8, or any combination thereof. In another embodiment, the anti-inflammatory response is elicited by upregulating the expression of IL-10.

In one embodiment, the RNA targeted in the above use of a composition can be a small RNA, a messenger RNA (mRNA), a ribosomal RNA (rRNA), a transfer RNA (tRNA), a small nuclear RNA (snRNA), a regulatory RNA, a transfer-messenger RNA (tmRNA), a double-stranded RNA (dsRNA), or a combination thereof. In one embodiment, the RNA is 16S ribosomal RNA or 23S ribosomal RNA.

In one embodiment, the RNA inhibitor used in the above use of a composition is RNase. In one embodiment, the RNA inhibitor can be RNase I, RNase A, RNase H, RNase III, RNase L, RNase P, RNase PhyM, RNase T1, RNase T2, RNase U2, RNase V, nuclease P1, micrococcal nuclease, PNPase, RNase PH, RNase R, RNase D, RNase T, oligoribonuclease, exoribonuclease I, or exoribonuclease II. In another embodiment, the RNA inhibitor is an endonuclease capable of cleaving tRNA in a virulent strain of acne bacterium. In another embodiment, the RNA inhibitor is tRNase or tRNA cleaving RNAse. In yet another embodiment, the RNA inhibitor is VapC toxin.

In one embodiment, the RNA inhibitor is administered by topical administration, trans-dermal administration, or subcutaneous administration. In another embodiment, the RNA inhibitor is co-administered with another agent. In one embodiment, this another agent can be an acne treating agent. Examples of an acne treating agent include, but are not limited to, benzoyl peroxide, a retinoid, an antibiotic, a hormonal agent, azelaic acid, salicylic acid, or a combination thereof. In one embodiment, the RNA inhibitor is administered independently from this another agent. In another embodiment, the RNA inhibitor is administered in combination with an acne treatment procedure. The acne treatment procedure can be comedo extraction, light therapy, dermabrasion, microneedling. chemical peel, or a combination thereof.

In one embodiment, the acne treated in the above use of a composition can be Acne vulgaris, Acne inversa, or Acne rosacea. In another embodiment, the acne is associated with Cutibacterium acnes, Staphylococcus epidermidis, Cutibacterium granulosum, or Cutibacterium humerusii. In one embodiment, the subject treated in the above use of a composition is a mammal. In another embodiment, the subject is a human.

In another embodiment, the present disclosure provides use of a composition for the preparation of medicament to treat a skin inflammation induced by Cutibacterium acnes in a subject in need thereof, the composition comprises a therapeutically effective amount of an RNA inhibitor.

In one embodiment, the RNA inhibitor in the methods or uses mentioned above is co-administered to a subject with an agent that downregulates the expression and/or function of one or more of IFN-γ, TNF-α, IL-1β, IL-6, IL-8, IL-17, IL-18, IL-18R, and TLR-8. In one embodiment, the RNA inhibitor can be co-administered with an agent that downregulates the expression and/or function of IFN-γ. In one embodiment, the RNA inhibitor can be co-administered with an agent that downregulates the expression and/or function of TNF-α. In one embodiment, the RNA inhibitor can be co-administered with an agent that downregulates the expression and/or function of IL-1β. In one embodiment, the RNA inhibitor can be co-administered with an agent that downregulates the expression and/or function of IL-6. In one embodiment, the RNA inhibitor can be co-administered with an agent that downregulates the expression and/or function of IL-8. In one embodiment, the RNA inhibitor can be co-administered with an agent that downregulates the expression and/or function of IL-17. In one embodiment, the RNA inhibitor can be co-administered with an agent that downregulates the expression and/or function of IL-18. In one embodiment, the RNA inhibitor can be co-administered with an agent that downregulates the expression and/or function of IL-18R. In one embodiment, the RNA inhibitor can be co-administered with an agent that downregulates the expression and/or function of TLR-8. In one embodiment, the RNA inhibitor can be co-administered with an agent that downregulates the expression and/or function of IL-18 and an agent that downregulates the expression and/or function of TLR-8.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1

### Treating Acne by the Use of RNA Inhibitors

Acne is a chronic inflammatory skin disease thought to be driven in part by the activation of the immune response to Cutibacterium acnes (C. acnes), a ubiquitous skin commensal. It has been reported that the relative abundance of C. acnes is similar in the follicles of acne patients compared to controls. This indicates that other characteristics of the bacteria play a role in how the skin reacts to C. acnes. Metagenomic analyses of the skin microbiome revealed that C. acnes is the dominant species in the pilosebaceous unit while commensals such as Staphylococcus epidermidis, Cutibacterium granulosum, and Cutibacterium humerusii makeup a smaller proportion. Phylogenetic investigation demonstrated that C. acnes isolated from the skin of acne patients compared to those with clear skin exhibit genetic and phenotypic differences. Specifically, strains of C. acnes found to be enriched on the skin of acne patients (C_{A}) induces higher inflammatory IFN-γ, TNF-α, and IL-1β secretion from human peripheral blood mononuclear cells (PBMCs) while strains were found to be associated with clear or healthy skin (C_{H}) induce higher anti-inflammatory IL-10. We set out to investigate the mechanisms of how the immune system distinguishes different strains of C. acnes to activate or dampen the inflammatory response. Exposure of PBMCs to live C. acnes led to the robust early production of IFN-γ as compared to tetanus toxoid, a known adaptive immune activator, suggesting an innate immune response. The IFN-γ response was abrogated when C. acnes was heat-killed as well as when treated with RNase while no reduction was observed in DNase-treated bacteria. RNA from C_{A} was found to induce higher inflammatory IFN-γ while RNA from C_{H} was found to induce higher IL-10 in both PBMCs and MDMs (monocyte-derived macrophages); a similar pattern seen with live bacteria. Bioanalyzer analysis reveals a striking difference between the C_{AN RNA} profile compared to those from C_{H}. CAN RNA displays a 25-200 nucleotide (nt) size peak while C_{H} and commensal strain traces have peaks at ranging from 25-4000 nt. This suggests C_{A} has a bacterial tRNA species that dominates while C_{H} and commensal strains have ribosomal RNA that C_{A} lacks. Our results show the use of RNase or inhibitor of RNA as a therapeutic option for acne.

### Materials and Methods:

Cells purification and culture. Whole blood was obtained from healthy donors who provided written informed consent (UCLA Institutional Review Board). PBMCs were isolated by Ficoll-Paque (Amersham Bioscience) density gradient centrifugation and cultured in RPMI 1640 (Gibco) supplemented with 10% fetal bovine serum (FBS) (Seradigm) at a density of 2.5×10⁶/mL in a 24-well flat bottomed plate.

Macrophages were generated from CD14 positive cells isolated using CD14 microbeads (Miltenyi Biotec) according the manufacture protocol. CD14 positive cells were cultured with M-CSF (50 ng/mL) or GM-CSF (50 ng/mL) in RPMI 1640 supplemented with 10% FBS for 5-6 days at a density of 5×10⁵/mL in a 24-well plate.

Reagents for Cell Stimulation. 19 kD, a TLR2/1L synthetic lipopeptide derived from the 19 kDa mycobacterial lipoprotein was obtained from EMC Microcollections and used at 10 ug/ml. LPS E. coli (Sigma) was used at a concentration of 2 μg/ml. LTA-SA (TLR2 agonist), ssRNA40/LyoVec (TLR8 agonist), and TL8-506 (TLR8 agonist) (Invivogen) were used according to manufacturer protocol.

Bacteria. C. acnes were obtained from BEI resources and include HL005PA1, HL043PA1, HL096PA1, HL042PA3, HL110PA3, HL110PA4. Colonies were grown on Brucella agar with 5% sheep blood, hemin, and vitamin K (Thermo Fisher Scientific Remel Products, Lenexa, Kans.) at 37 C for 5-7 days under anaerobic conditions in sealed containers containing oxygen-absorbing carbon dioxide-generating Aaero Packs (Mitsubishi Gas Chemical Co., Inc, Tokyo, Japan), Cultures inoculated from single colonies were grown under the same conditions in Reinforced Clostridial Medium (Oxoid, Basingstoke, England).

Bacterial treatment. Live bacteria were heat-killed at 95° C. for 10 minutes and cooled on ice before addition of bacteria to cell culture. Live bacteria were digested using RNase I (Promega) or DNase I (Invitrogen) and incubated at 37° C. for 1 hour. Nucleases were inactivated with 0.1% SDS.

Bacterial total RNA extraction. Total RNA from C. acnes pellets was extracted as described [11, 12] using phenol:chloroform:isoamyl alcohol and sodium acetate pH 5.2. Aqueous phase was collected and total RNA precipitated with 1:1 isopropanol at −20° C. for 1 hour. RNA pellet was washed with ethanol, air-dried, and resuspended in RNase-free water. RNA profiles were analyzed on RNA 6000 Nano chips using Agilent Bioanalyzer (Agilent Technologies) at the Technology Center for Genomics & Bioinformatics (TCGB) Core at UCLA. All RNA samples were used immediately or stored at −80 C.

Cytokine Quantification. Cell culture supernatants were harvested at 24 hours unless otherwise noted. Cytokines measured by sandwich ELISA using antibody pairs were as follows: IL-18, IL-1β, IFN-β (R&D Duoset), IFN-γ (BD), IL-6, IL-10, TNF-α (Invitrogen).

### Results:

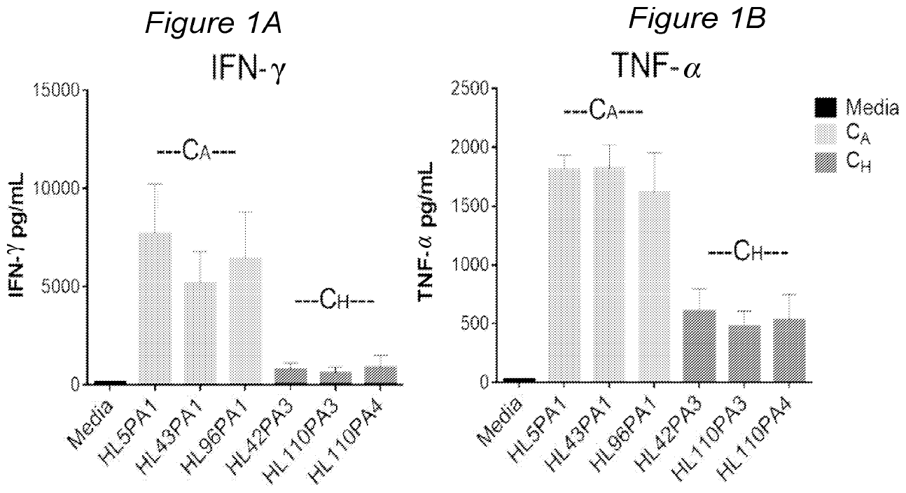

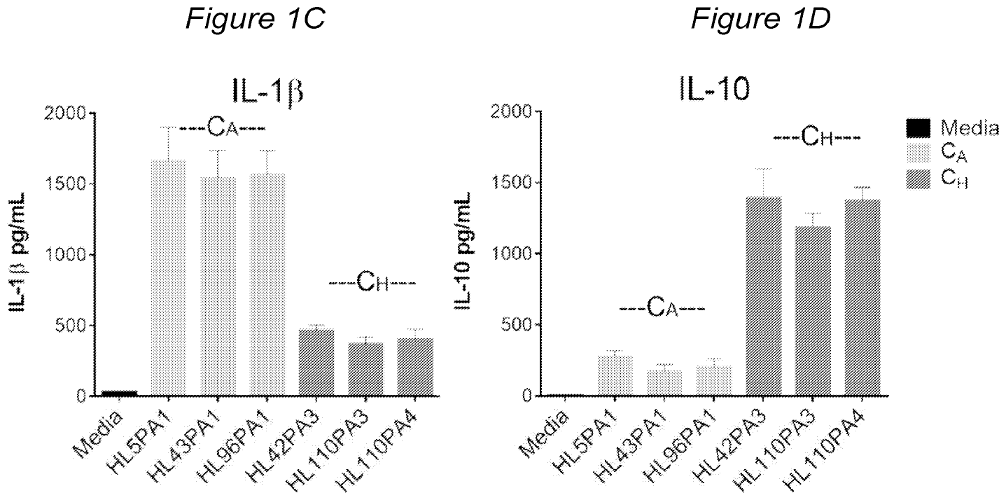

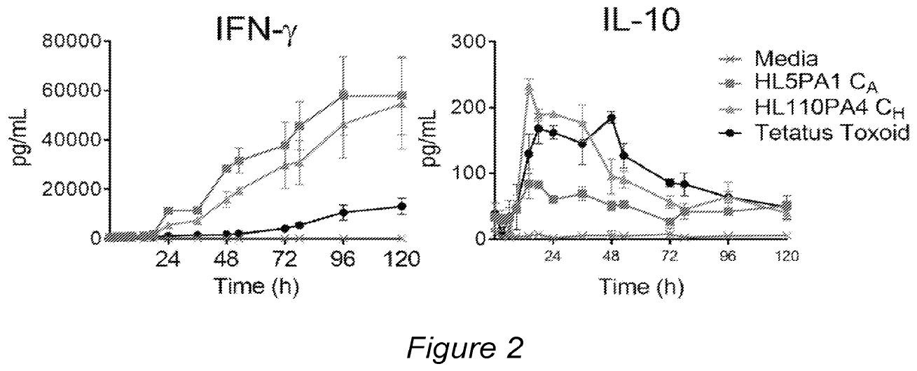

C_{A} vs. C_{H} induce distinct immune response. Our previous work demonstrated that C_{A} induce higher IFN-γ and IL-17, pro-inflammatory cytokines produced by Th1 and Th17 cells suggesting C_{A} plays a role in activating the adaptive immune response. In contrast, C_{H} induce higher anti-inflammatory cytokine IL-10. Here it is found that C_{A} induce higher IFN-γ, TNF-α, and IL-1β while C_{H} induce higher IL-10 at 24 hours (FIG. 1). A time course of IFN-γ and IL-10 induction revealed that both cytokines are induced early suggesting a mechanism of innate immune activation (FIG. 2).

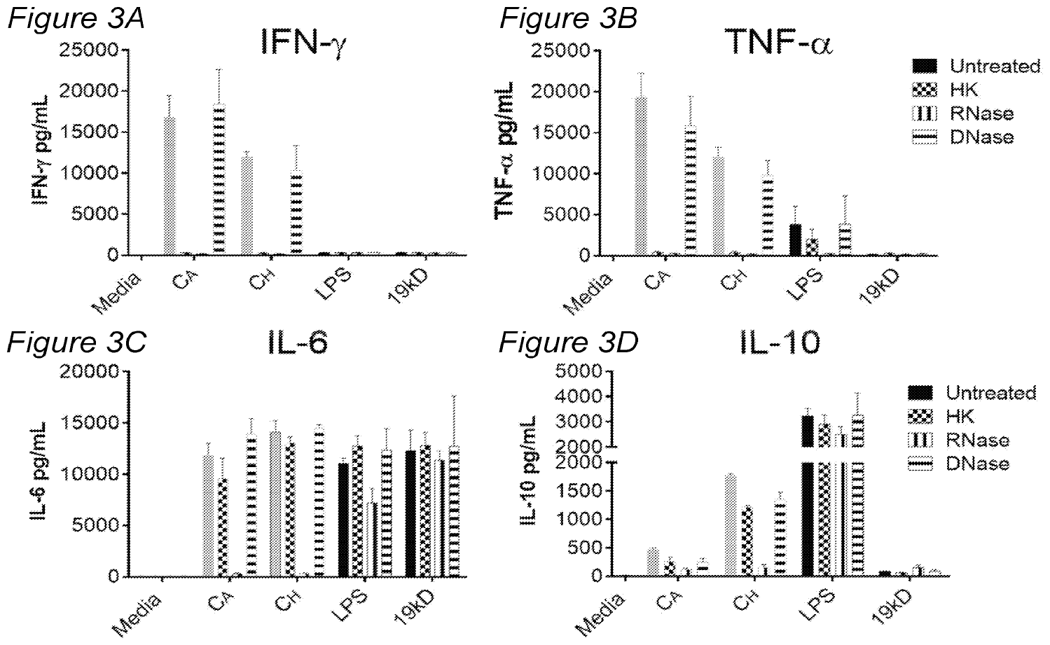

RNase treatment inhibited inflammatory cytokine secretion. The addition of RNase I to live bacteria eliminated IFN-γ, TNF-α, IL-6, and IL-10 secretion from PBMCs, whereas DNase I treatment had no effect (FIG. 3). Interestingly, heat-killed bacteria induced IL-6 and IL-10 (FIG. 3C-D) but not IFN-γ nor TNF-α secretion (FIG. 3A-B).

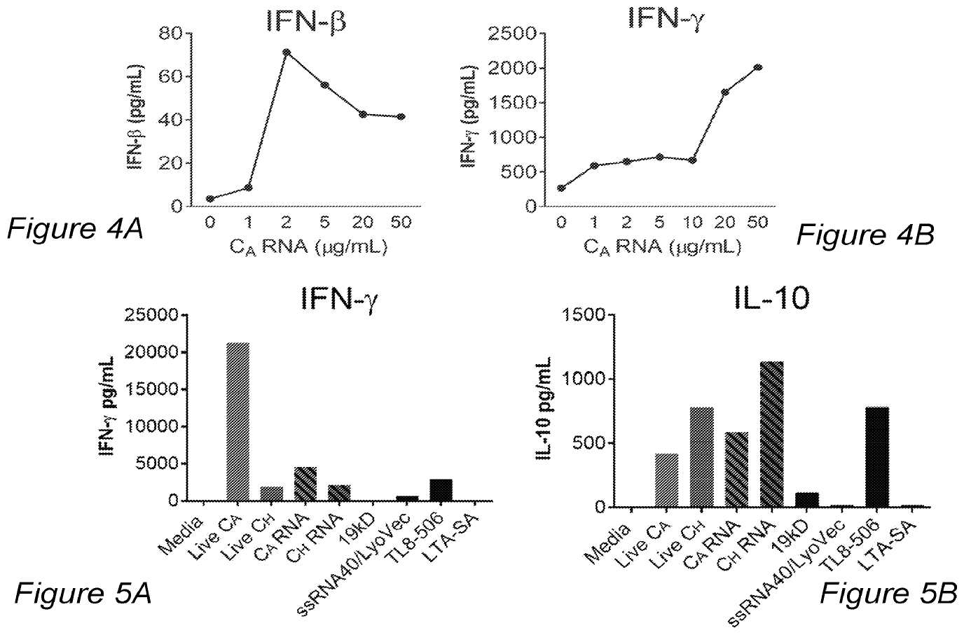

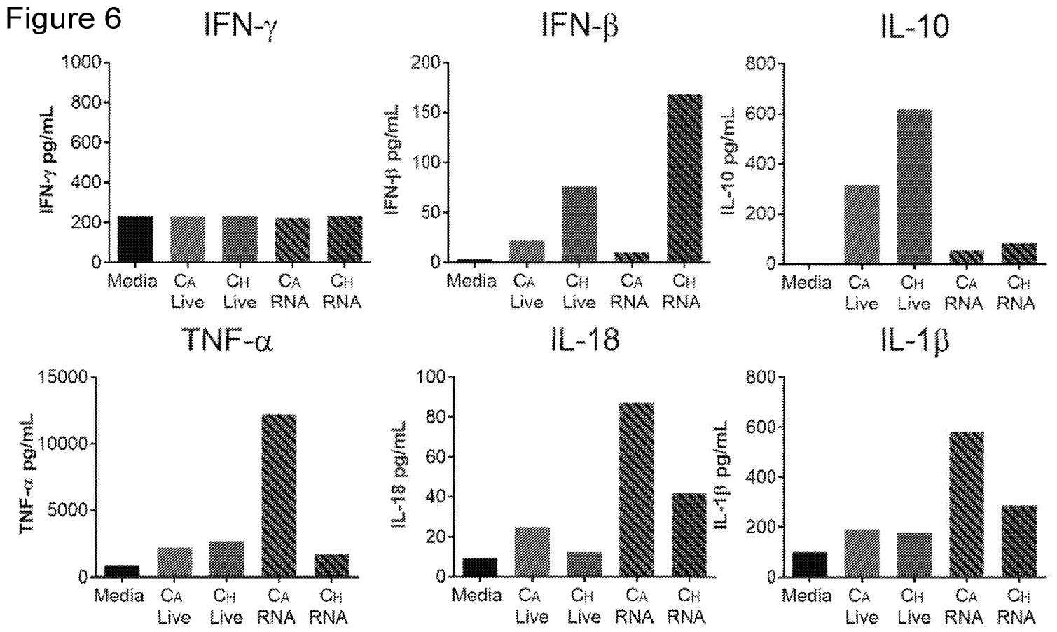

C. acnes RNA induced similar effects to live bacteria. These findings raised the question whether RNA plays a role in C. acnes-induced inflammation. To address this question, total RNA was isolated from C. acnes cultures. It is found total bacterial C_{AN RNA} induces IFN-β and IFN-γ secretion from PBMCs in a dose-dependent manner (FIG. 4). Both live C_{A} and total RNA isolated from C_{A} induce higher IFN-γ while live C_{H} and total RNA isolated from C_{H} induce higher IL-10 secretion from PBMCs (FIG. 5). It is observed the same distinct cytokine responses in MDMs that differentiate C_{A} and C_{H} in both live bacteria and total RNA (FIG. 6).

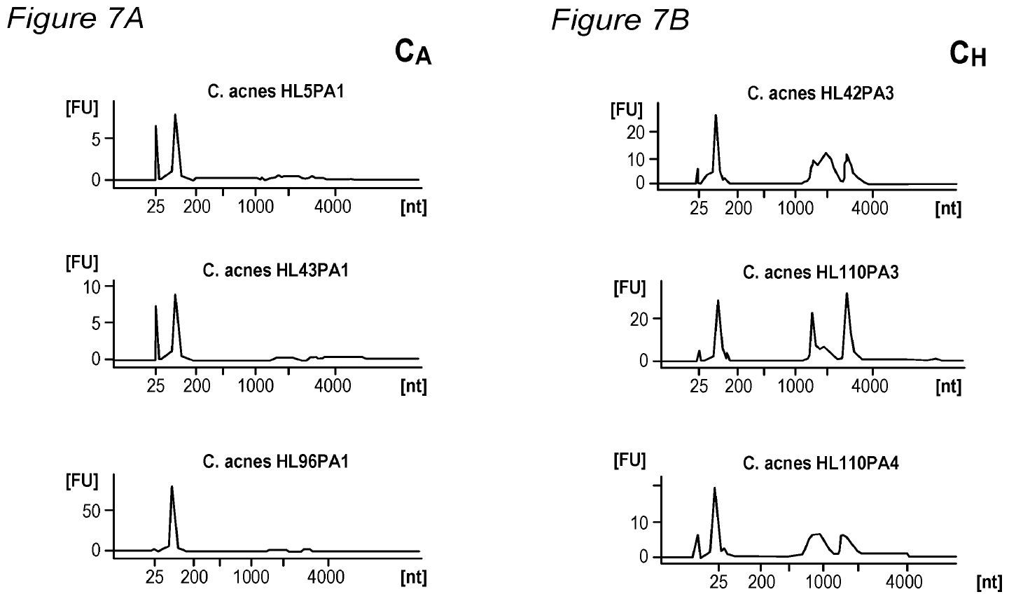

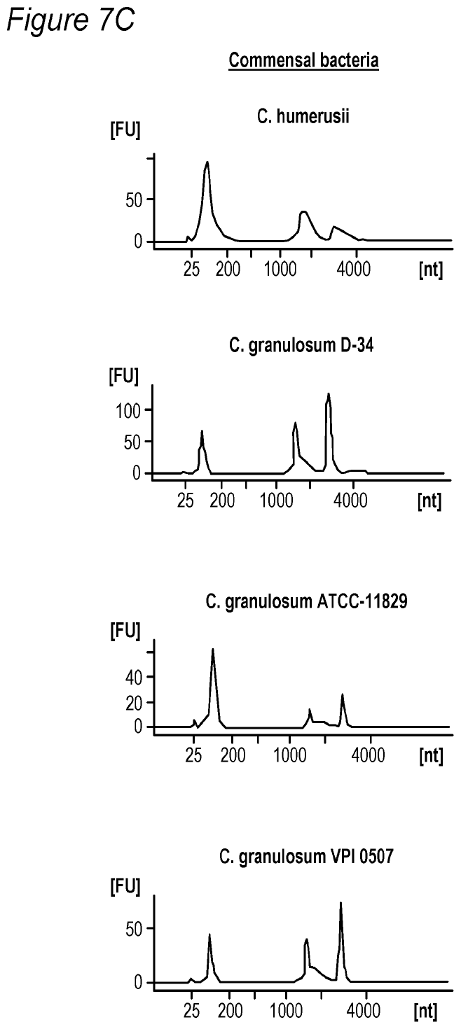

RNA species from C_{A} is dominated by tRNA. In order to characterize total RNA obtained from C. acnes, samples were analyzed using the Bioanalyzer RNA 6000 Nano assay. The bioanalyzer traces of the sample showed C_{AN RNA} predominantly ranges from 25-200 nucleotides (nt) while C_{H} RNA has peaks from 25-200 nt as well as peaks from 1000-4000 nt (FIG. 7A-B). This suggests C_{A} has low levels of rRNA (1000-4000 nt) or that its tRNA species (25-200 nt) is produced in excess compared to rRNA (FIG. 7A). It is observed that the bioanalyzer traces in other commensal inhabitants of the pilosebaceous gland such as: C. humerusii, and C. granulosum were similar to C_{H} (FIG. 7C).

In summary, these results show the use of an RNAse or an inhibitor of RNA as a therapeutic option for acne.

### Example 2

### Cytokine Expression in Acne

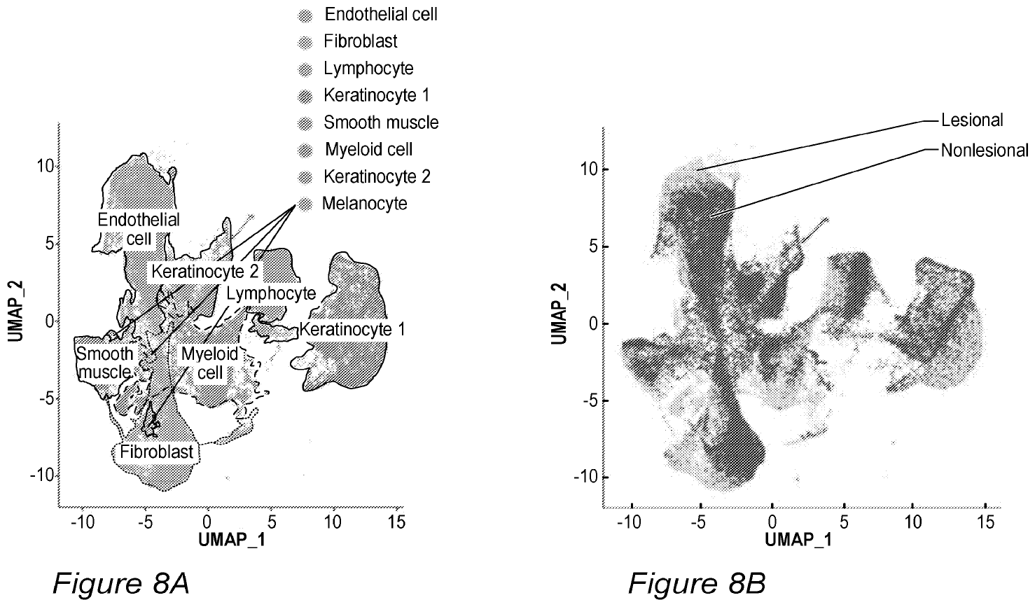

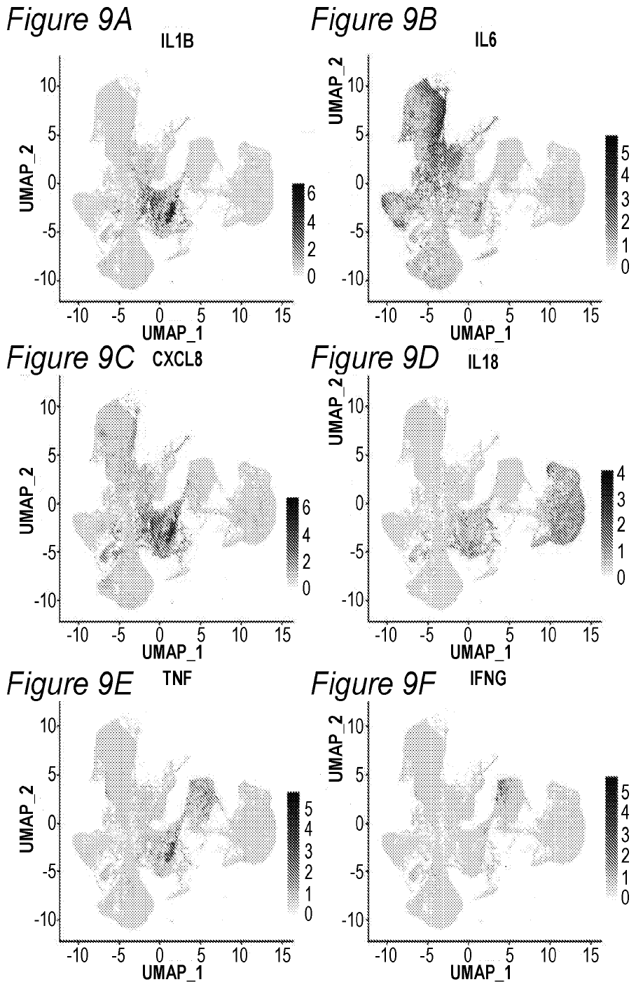

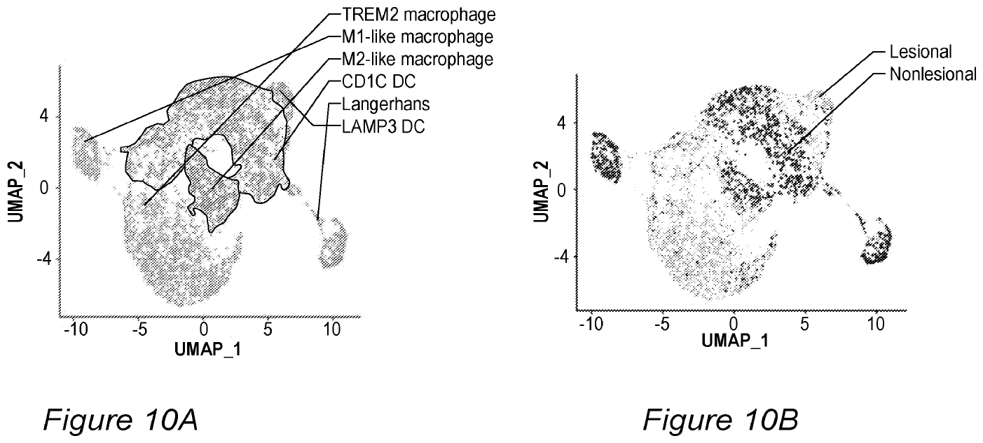

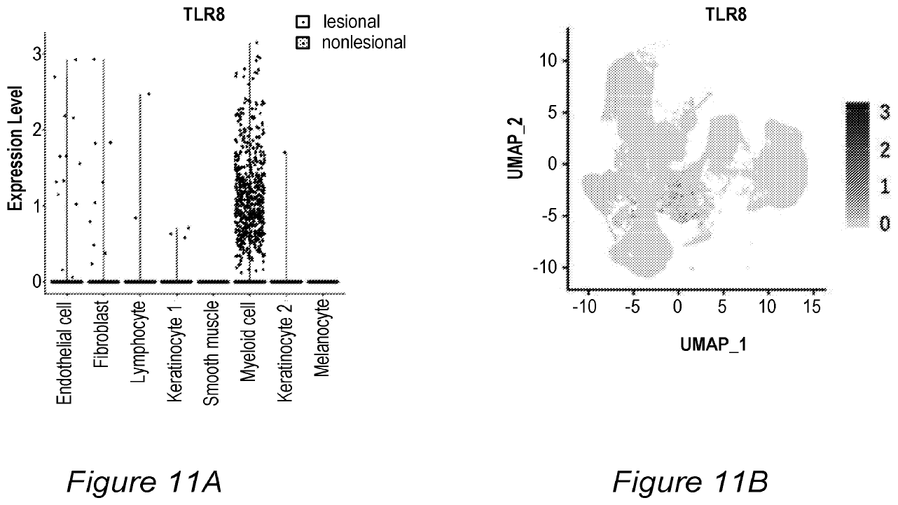

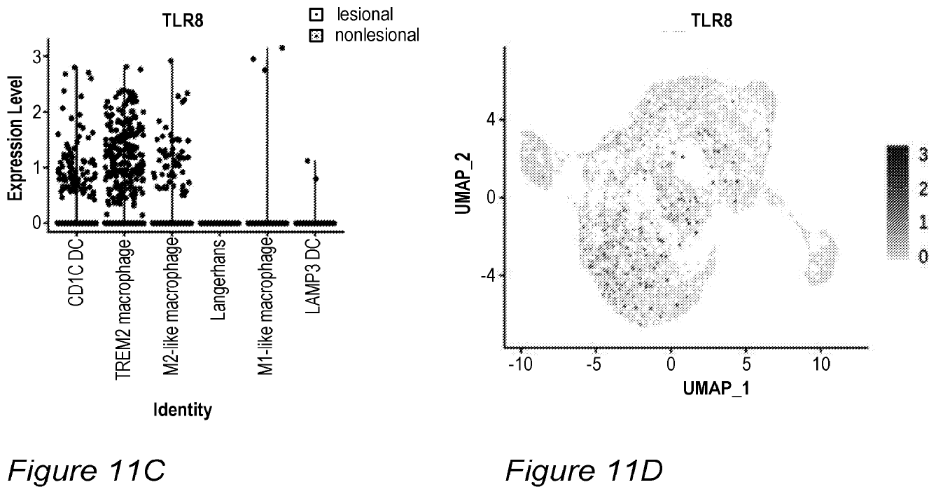

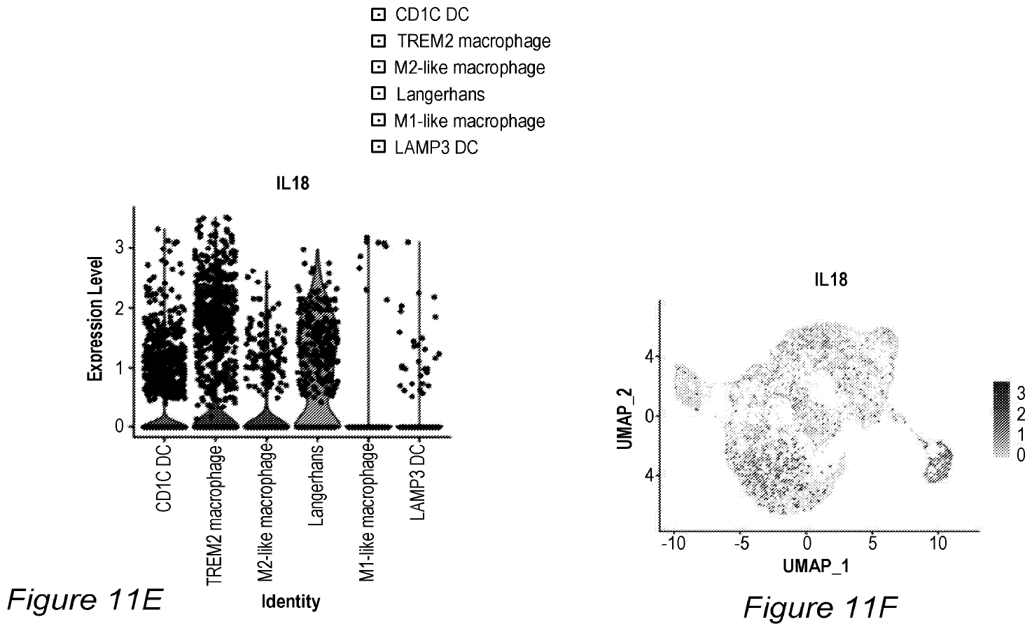

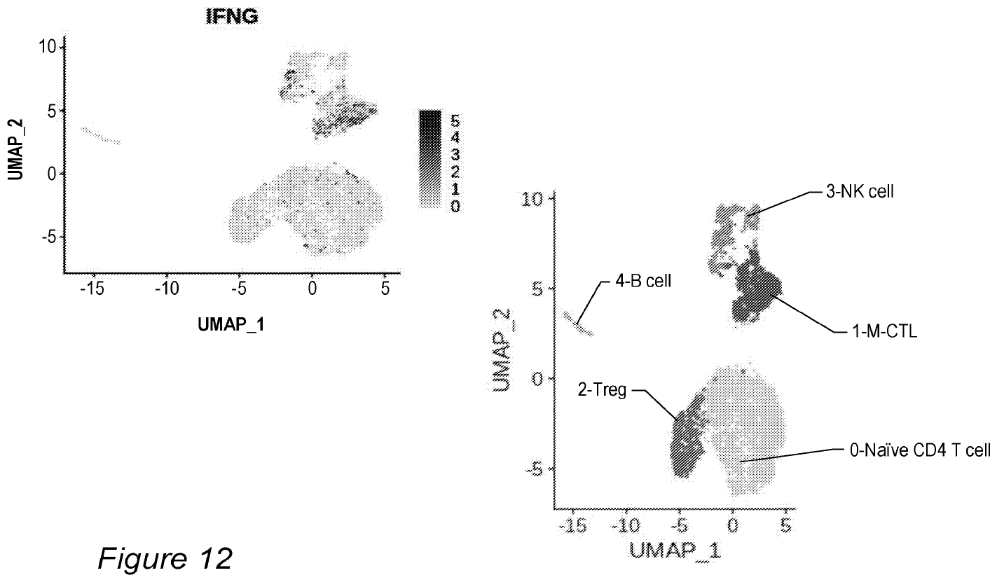

Single-cell RNA sequencing (scRNA-seq) has emerged as a powerful tool to deconstruct genetic and molecular profiles of individual cells, requiring few cells for analysis, such that it is readily possible to study immune cells from disease lesions. scRNA-seq analysis was performed on papules and non-lesional skin from acne patients to dissect the immune mechanisms contributed by specific cell populations to disease immune pathogenesis. Different cell clusters were classified using differentially expressed genes and established canonical markers to manually assign cell-types. TLR8, IL-8 (CXCL8), and IL-18 expression were identified in the myeloid population with IL-18 expression higher in lesional compared to non-lesional skin (FIGS. 8-9). This is consistent with in vitro data that C. acnes from acne lesions is a potent inducer of IL-18. These data suggest that TLR8 and IL-18 are expressed in TREM2-expressing macrophages; these cells have been implicated in disorders of lipid metabolism such as atherosclerosis and obesity (FIGS. 10-11). It was also found that lymphocytes, specifically NK cells and mono-cytotoxic lymphocytes, were the main producers of IFN-γ, consistent with in vitro data (FIG. 9F, FIG. 12). TNF-α is highly expressed in myeloid cells and lymphocytes in the lesions which is consistent with its known functions to drive the recruitment of inflammatory cells (see FIG. 9E). IL-6 expression is upregulated in endothelial cells and smooth muscle which could be related to vasodilation that gives inflammatory acne lesions their bright red color (see FIG. 9B). These findings provide new opportunities to dive deep and re-examine the pathogenesis of acne and potentially identify novel targets for therapy.

What is claimed is:

1. Use of a composition for the preparation of medicament to treat acne in a subject in need thereof, the composition comprises a therapeutically effective amount of an RNA inhibitor.

2. The use of claim 1, wherein said RNA inhibitor inhibits or degrades RNA in a virulent strain of acne bacterium.

3. The use of claim 1, wherein said RNA inhibitor elicits an anti-inflammatory response.

4. The use of claim 3, wherein said anti-inflammatory response is elicited by downregulating the expression of IFN-γ, TNF-α, IL-1β, IL-6, IL-8, IL-17, IL-18, IL-18R, TLR-8, or any combination thereof.

5. The use of claim 3, wherein said anti-inflammatory response is elicited by upregulating the expression of IL-10.

6. The use of claim 2, wherein said RNA is a small RNA, a messenger RNA (mRNA), a ribosomal RNA (rRNA), a transfer RNA (tRNA), a small nuclear RNA (snRNA), a regulatory RNA, a transfer-messenger RNA (tmRNA), a double-stranded RNA (dsRNA), or a combination thereof.

7. The use of claim 2, wherein said RNA is 16S ribosomal RNA or 23S ribosomal RNA.

8. The use of claim 1, wherein said RNA inhibitor is RNase.

9. The use of claim 1, wherein said RNA inhibitor is RNase I, RNase A, RNase H, RNase III, RNase L, RNase P, RNase PhyM, RNase T1, RNase T2, RNase U2, RNase V, nuclease P1, micrococcal nuclease, PNPase, RNase PH, RNase R, RNase D, RNase T, oligoribonuclease, exoribonuclease I, or exoribonuclease II.

10. The use of claim 1, wherein said RNA inhibitor is an endonuclease capable of cleaving tRNA in a virulent strain of acne bacterium.

11. The use of claim 1, wherein said RNA inhibitor is tRNase or tRNA cleaving RNAse.

12. The use of claim 1, wherein said RNA inhibitor is VapC toxin.

13. The use of claim 1, wherein said RNA inhibitor is administered by topical administration, trans-dermal administration, or subcutaneous administration.

14. The use of claim 1, wherein said RNA inhibitor is co-administered to the subject with another agent.

15. The use of claim 14, wherein said another agent is an acne treating agent.

16. The use of claim 15, wherein said acne treating agent is benzoyl peroxide, a retinoid, an antibiotic, a hormonal agent, azelaic acid, salicylic acid, or a combination thereof.

17. The use of claim 14, wherein the RNA inhibitor is administered independently from said another agent.

18. The use of claim 14, wherein said another agent is an agent that downregulates the expression or function of one or more of IFN-γ, TNF-α, IL-1β, IL-6, IL-8, IL-17, IL-18, IL-18R, and TLR-8.

19. The use of claim 18, wherein the RNA inhibitor is co-administered to the subject with an agent that downregulates the expression or function of IL-18.

20. The use of claim 18, wherein the RNA inhibitor is co-administered to the subject with an agent that downregulates the expression or function of TLR-8.

21. The use of claim 1, wherein said RNA inhibitor is administered to the subject in combination with an acne treatment procedure.

22. The use of claim 21, wherein said acne treatment procedure is comedo extraction, light therapy, dermabrasion, microneedling. chemical peel, or a combination thereof.

23. The use of claim 1, wherein said acne is Acne vulgaris, Acne inversa, or Acne rosacea.

24. The use of claim 1, wherein said acne is associated with Cutibacterium acnes, Staphylococcus epidermidis, Cutibacterium granulosum, or Cutibacterium humerusii.

25. The use of claim 1, wherein said subject is a mammal.

26. The use of claim 1, wherein said subject is a human.

27. Use of a composition for the preparation of medicament to treat a skin inflammation induced by Cutibacterium acnes in a subject in need thereof, the composition comprises a therapeutically effective amount of an RNA inhibitor.
